(19) United States
(12) Patent Application Publication
(10) Pub. No.: US 2025/0136912 A1
(43) Pub. Date: May 1, 2025

(54) CELL CULTURE MEMBRANE AND MEASUREMENT METHOD

(71) Applicants: TOYODA GOSEI CO., LTD., Kiyosu-shi (JP); TOHOKU UNIVERSITY, Sendai-shi (JP)

(72) Inventors: Sayaka MIYATA, Aichi (JP); Yasuhiko SHINODA, Aichi (JP); Seitaro TAKI, Aichi (JP); Kosuke INO, Miyagi (JP); Yoshinobu UTAGAWA, Miyagi (JP); Hitoshi SHIKU, Miyagi (JP)

(21) Appl. No.: 18/930,711

(22) Filed: Oct. 29, 2024

(30) Foreign Application Priority Data
Oct. 30, 2023 (JP) 2023-185736

(51) Int. Cl. C12M 1/12; C12M 1/34

(52) U.S. Cl. CPC C12M 25/02; C12M 41/32

(57) ABSTRACT

A cell culture membrane includes: a membrane body having a first face, a second face located on a side opposite to the first face, and plural through holes penetrating from the first face to the second face; and a metal film including at least one of a first metal film provided to overlap the first face or a second metal film provided to overlap the second face, and the plural through holes have a first average hole diameter at the first face larger than a second average hole diameter at the second face.

### CROSS-REFERENCE TO RELATED APPLICATION

This application is based on and claims priority under 35 USC 119 from Japanese Patent Application No. 2023-185736 filed on Oct. 30, 2023.

### TECHNICAL FIELD

The present disclosure relates to a cell culture membrane and a measurement method.

### BACKGROUND ART

Conventionally, in order to evaluate the state of cell culture, measurement of a physical property value of a culture solution and measurement of the amount of a specific substance in the culture solution are performed (for example, JP2020-146015A). JP2020-146015A describes a device for measuring trans-epithelial electrical resistance using electrodes. In the cell culture, as the measurement using the electrodes, impedance and pH of the culture solution, the amount of glucose, and the like may be measured in addition to the trans-epithelial electrical resistance.

### SUMMARY OF INVENTION

Since the above measurement is the measurement of the culture solution, a measurement error may easily increase depending on the arrangement pattern of the electrodes.

The present disclosure can be implemented by the following aspects.

(1) According to a first aspect of the present disclosure, a cell culture membrane is provided. The cell culture membrane includes: a membrane body having a first face, a second face located on a side opposite to the first face, and a plurality of through holes penetrating from the first face to the second face; and a metal film including at least one of a first metal film formed to overlap the first face or a second metal film formed to overlap the second face, in which the plurality of through holes have a first average hole diameter at the first face larger than a second average hole diameter at the second face. According to this aspect, when cells are cultured on a metal film, since the metal film is in contact with or close to the cells in the measurement using the metal film as an electrode, a measurement accuracy of a culture state in the vicinity of the cells can be improved. Further, when different types of cells are cultured on the first face and the second face, respectively, since the cells cultured on the first face and the cells cultured on the second face can come into contact with each other through the through holes, a cellular condition closer to that in a living body can be created.

(2) In the cell culture membrane according the above aspect, each of the plurality of through holes may have a first opening portion opening at the first face, a second opening portion opening at the second face, and an inner peripheral portion connecting the first opening portion to the second opening portion, the metal film may include the second metal film and include the first metal film, and the first metal film may have a metal inner peripheral portion formed to cover the inner peripheral portion. According to this aspect, since it is possible to increase an area of the metal film in contact with or close to the cells that have entered the through holes, a measurement accuracy can be further improved.

(3) In the cell culture membrane according to the above aspect, the first metal film may have a metal closing portion which closes the second opening portion, and the metal closing portion may have a crack. According to this aspect, the cells cultured on the first face and the cells cultured on the second face can come into contact with each other through a crack, and since the size of the crack is smaller than the size of the second opening portion, it is expected that the cellular condition closer to that in the living body can be created.

(4) In the cell culture membrane according to the above aspect, the membrane body may be formed of polyurethane. According to this aspect, it is possible to easily produce the membrane body having the plurality of through holes whose first average hole diameter is larger than the second average hole diameter at the second face.

(5) In the cell culture membrane according to the above aspect, the second average hole diameter may be 7 μm or less. According to this aspect, when cells having a size of about 10 μm are cultured, it is possible to prevent the cells cultured on the first face or the second face from moving to the other of the first face and the second face through the through holes.

(6) In the cell culture membrane according to the above aspect, the metal film may include a first metal layer mainly made of either gold (Au) or platinum (Pt). According to this aspect, the first metal layer mainly made of gold or the first metal layer mainly made of platinum can be used as the metal film.

(7) The cell culture membrane according to the above aspect may further include: a titanium layer disposed between the first metal layer and the membrane body and mainly made of titanium (Ti). According to this aspect, adhesion of the first metal layer to the membrane body can be improved by the titanium layer. Thus, the measurement accuracy can be improved.

(8) In the cell culture membrane according to the above aspect, the metal film may include a first metal layer mainly made of gold (Au), and a second metal layer mainly made of platinum (Pt) and disposed between the first metal layer and the membrane body. According to this aspect, the measurement accuracy can be improved.

(9) In the cell culture membrane according to the above aspect, the metal film may further include a titanium layer disposed between the second metal layer and the membrane body and mainly made of titanium (Ti). According to this aspect, adhesion of the first metal layer and the second metal layer to the membrane body can be improved by the titanium layer. Thus, the measurement accuracy can be improved.

(10) A measurement method using the cell culture membrane according to the above aspect may include: a first step of immersing the cell culture membrane in a culture solution to culture a cell on the metal film of the cell culture membrane; and a second step of electrochemically measuring a glucose concentration using the metal film as a working electrode in the culture solution. According to this aspect, when the cells are cultured on the cell culture membrane, the measurement of the glucose concentration using the metal film as the electrode can be achieved.

(11) A measurement method using the cell culture membrane according to the above aspect may include: a first step of immersing the cell culture membrane in a culture solution to culture a cell on the metal film of the cell culture membrane; and a second step of performing electrochemical impedance measurement using the metal film as a working electrode in the culture solution. According to this aspect, when the cells are cultured on the cell culture membrane, electrochemical impedance measurement using the metal film as the electrode can be achieved. The present disclosure can be implemented in various aspects, and can be implemented in the aspect of, for example, a manufacturing method of a cell culture membrane in addition to the above cell culture membrane.

### BRIEF DESCRIPTION OF DRAWINGS

FIG. 1 is a schematic diagram of a cross section of a cell culture membrane.

FIG. 2 is a flowchart showing a manufacturing process of a cell culture membrane.

FIG. 3 is a flowchart showing a procedure of a measurement method of measuring a glucose concentration.

FIG. 4 is a view showing a configuration of a measurement system.

FIG. 5 shows a measurement result obtained by measuring a relationship between the glucose concentration and a current value.

FIG. 6 is a schematic diagram in a case of co-culturing using the cell culture membrane.

FIG. 7 is a flowchart showing a procedure of a measurement method of measuring impedance.

FIG. 8 is a view showing a measurement system for performing impedance measurement using three electrodes.

FIG. 9 shows views of other embodiments of the cell culture membrane.

FIG. 10 shows SEM images obtained by observing the cell culture membrane on which cells are cultured.

FIG. 11 is a diagram showing measurement results of the glucose concentration.

### DESCRIPTION OF EMBODIMENTS

### A. Embodiment

### A-1. Cell Culture Membrane

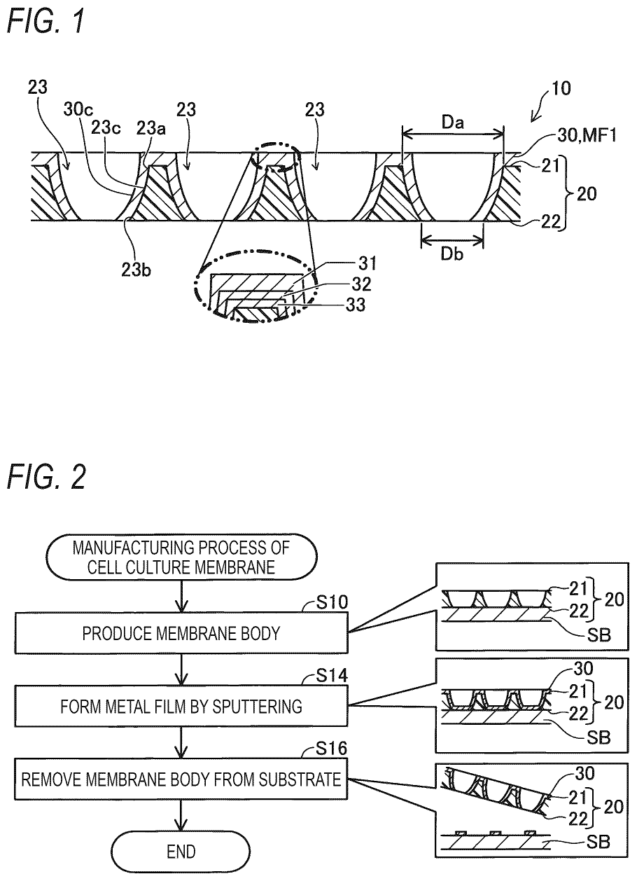

FIG. 1 is a schematic diagram of a cross section of a cell culture membrane 10. The cell culture membrane 10 includes a membrane body 20 and a metal film 30. In the present embodiment, the cell culture membrane 10 is formed of polyurethane. The metal film 30 is implemented by stacking a first metal layer 31 mainly made of gold (Au), a second metal layer 32 mainly made of platinum (Pt), and a titanium layer 33 mainly made of titanium (Ti) in this order from an upper layer toward a lower layer. That is, the titanium layer 33 is disposed between the second metal layer 32 and the membrane body 20. Since the titanium layer 33 is disposed between the second metal layer 32 and the membrane body 20, adhesion of the first metal layer 31 and the second metal layer 32 to the membrane body 20 can be improved.

In another embodiment of the metal film 30, the metal film 30 may include only the first metal layer. In this case, the first metal layer may be either a layer mainly made of gold or a layer mainly made of platinum (Pt). In this case, the titanium layer 33 may be further disposed between the first metal layer and the membrane body 20 in the metal film 30. The adhesion between the first metal layer 31 and the membrane body 20 can be improved by disposing the titanium layer 33. Further, in the present embodiment, the metal film 30 includes the first metal layer 31, the second metal layer 32, and the titanium layer 33 stacked in this order. In another embodiment, the titanium layer 33 is not provided, and the metal film 30 may include only the first metal layer 31 and the second metal layer 32. Since the cell culture membrane 10 includes the metal film 30, various electrical measurements can be performed by culturing cells on the metal film 30 and then using the metal film 30 as an electrode.

The membrane body 20 has a first face 21, a second face 22 opposite to the first face 21, and a plurality of through holes 23 formed in the membrane body 20. The plurality of through holes 23 are holes penetrating from the first face 21 to the second face 22. Each through hole 23 has a first opening portion 23a opening at the first face 21, a second opening portion 23b opening at the second face 22, and an inner peripheral portion 23c connecting the first opening portion 23a to the second opening portion 23b. Since the cell culture membrane 10 has the second opening portions 23b, different types of cells can be brought into contact with each other when they are cultured on the first face 21 and the second face 22, respectively.

A hole diameter of the through hole 23 at the first face 21 is different from a hole diameter of the through hole 23 at the second face 22. The hole diameter of the through hole 23 at the first face 21 is referred to as a first hole diameter Da, and the hole diameter of the through hole 23 at the second face 22 is referred to as a second hole diameter Db. The first hole diameter Da is larger than the second hole diameter Db. A first average hole diameter, which is an average value of the first hole diameters Da of the plurality of through holes 23 at the first face 21, is different from a second average hole diameter, which is an average value of the second hole diameters Db at the second face 22. The first average hole diameter is larger than the second average hole diameter. As will be described later, a cell 300 can be cultured in a state in which the cell 300 has entered the interior of the through hole 23. The second average hole diameter is preferably 7 μm or less. Accordingly, as will be described later, when co-culture is performed using the cell culture membrane 10, after first cells 301 are seeded on the cell culture membrane 10, the first cells 301 can be prevented from moving to a face opposite to the seeded face after passing through the through hole 23. In the following description, the movement of the first cells 301 to the face opposite to the seeded face after passing through the through holes 23 may be referred to as “wrap-around”.

The first average hole diameter is a value obtained by irradiating the upward-facing first face 21 with light and performing observation with a microscope in a state in which the first face 21 is on the top and the second face 22 is on the bottom. Since the light is not reflected from the through hole 23, the through hole 23 is visually recognized in black. Specifically, circle equivalent diameters of all the through holes 23 observed in a specific visual field are measured and an average value of the measured values is obtained. The circle equivalent diameter refers to a diameter of a true circle corresponding to an area of the through hole 23. The second average hole diameter is similarly obtained. That is, the value is obtained by irradiating the upward-facing second face 22 with light and performing observation with a microscope in a state in which the second face 22 is on the top and the first face 21 is on the bottom.

The metal film 30 includes a first metal film MF1 formed to overlap the first face 21. The first metal film MF1 has a metal inner peripheral portion 30c. The metal inner peripheral portion 30c is formed to cover the inner peripheral portion 23c. Since the metal film 30 is formed to cover the inner peripheral portion 23c, as will be described later, it is possible to further improve accuracy when an electrical measurement is performed in the vicinity of the cell 300.

### A-2. Manufacturing Method of Cell Culture Membrane

FIG. 2 is a flowchart showing a manufacturing process of the cell culture membrane 10. As shown in FIG. 2, in step S10, the membrane body 20 formed of polyurethane is produced. Specifically, water vapor is supplied to an uncured polyurethane raw material formed in a thin plate shape on a substrate SB, and the uncured polyurethane raw material is cured while foaming. According to this manufacturing method, since the through hole 23 is formed by foaming, the cell culture membrane 10 in which the first hole diameter Da and the second hole diameter Db are different from each other can be easily manufactured.

In step S14, the metal film 30 is formed on the first face 21 of the membrane body 20 by sputtering. Specifically, the membrane body 20 formed on the substrate SB is placed in a vacuum chamber together with the substrate SB, and a metal is deposited on the first face 21 to form the metal film 30. In the present embodiment, sputtering time of the first metal layer 31 is three minutes. Sputtering time of the second metal layer 32 is one minute. Sputtering time of the titanium layer 33 is one minute. The sputtering time is not limited to the above.

In step S16, the membrane body 20 is removed from the substrate SB, and the manufacturing process ends. In the process of removing the membrane body 20 from the substrate SB, the metal film formed on the substrate SB is separated from the membrane body 20. Therefore, the second opening portion 23b of the membrane body 20 is not closed by the metal film.

### A-3. Measurement Method of Glucose

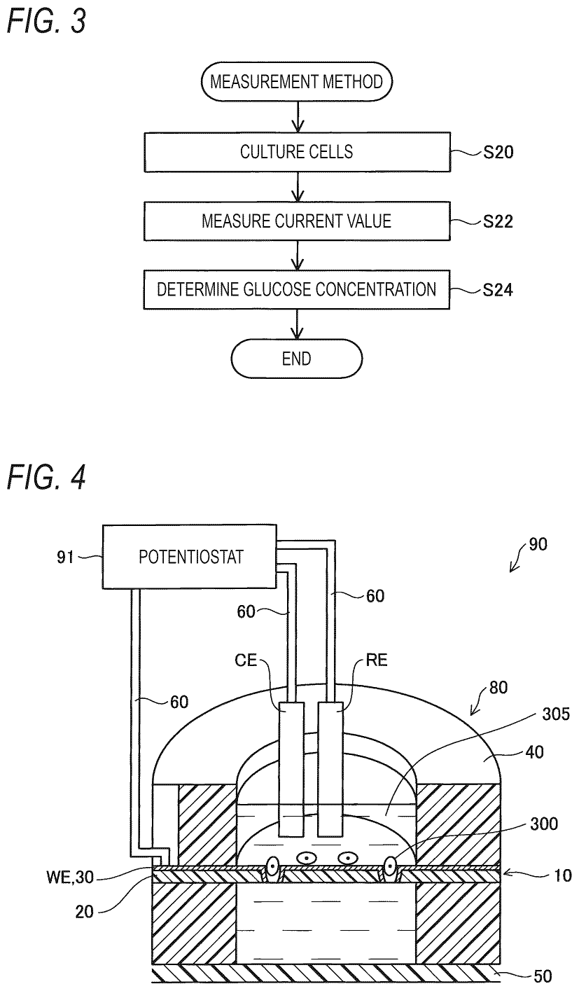

FIG. 3 is a flowchart showing a procedure of a measurement method of measuring a glucose concentration of the culture solution after the cells are cultured on the cell culture membrane 10. FIG. 4 is a view showing a configuration of a measurement system 90 assembled in the case of measuring the glucose concentration. Glucose is metabolized by the cells 300. Therefore, the culture state can be evaluated by measuring the glucose concentration of the culture solution in the culture step of the cells 300.

As shown in FIG. 3, in the culture step of step S20, the cells 300 are cultured on the cell culture membrane 10. In the present embodiment, culture is performed using a culture container 80 including the cell culture membrane 10.

As shown in FIG. 4, the culture container 80 includes a cell culture membrane 10 and a cylindrical member 40. The cylindrical member 40 is made of a resin and has a cylindrical shape. The cell culture membrane 10 is attached to a central position in an axial direction of the cylindrical member 40. The cylindrical member 40 is placed into a resin container 50 and used. A conductive wire 60 is electrically connected to the metal film 30 of the cell culture membrane 10. In the present embodiment, a thickness of the cell culture membrane 10 is 5 μm. In the present embodiment, an inner diameter of the cylindrical member 40 is 8 mm.

In step S20 as a first step shown in FIG. 3, in a state in which the culture container 80 shown in FIG. 4 is placed in the resin container 50, the culture solution 305 is placed inside the cylindrical member 40, and the cells 300 are seeded and cultured. The culture solution 305 contains the glucose.

As shown in FIG. 3, in step S22, a current value of a current flowing through the metal film 30 is measured. As shown in FIG. 4, the measurement system 90 includes a potentiostat 91, a counter electrode CE, and a reference electrode RE. The conductive wire 60 electrically connected to the metal film 30 is electrically connected to the potentiostat 91. In the measurement, the metal film 30 functions as a working electrode WE. The counter electrode CE and the reference electrode RE are electrically connected to the potentiostat 91 via the conductive wire 60.

In step S22, a potential difference between the metal film 30 functioning as the working electrode WE and the reference electrode RE is controlled to a preset potential difference by using the potentiostat 91 after pH of a surface of the working electrode WE is adjusted to be alkaline by a pretreatment. Specifically, the pretreatment is a treatment of generating hydroxide ions in the vicinity of the working electrode WE by causing an electrolysis reaction on the working electrode WE.

As shown in FIG. 3, in step S24, the glucose concentration in the culture solution 305 is determined using the measured current value. Specifically, the glucose concentration is determined using a relational expression determined in advance between the glucose concentration and the current value. Since the cell 300 is in contact with or close to the metal film 30, the glucose concentration in the vicinity of the cell 300 can be accurately measured.

Specifically, an approximate expression can be used as the relational expression between the glucose concentration and the current value. Alternatively, the glucose concentration may be determined using a map representing the relationship between a range of the current values and the glucose concentration applied to each range. Steps S22 and S24 are also referred to as a second step. As described above, in the second step, the glucose concentration is electrochemically measured using the metal film 30 as the working electrode in the culture solution 305.

### A-4. Measurement Principle of Glucose Concentration

Regarding an electrode reaction of glucose at a gold electrode in alkaline conditions, a proposal is made in “Nanostructured gold deposited in gelatin template applied for electrochemical assay of glucose in serum” by Tomáš Julik et al., Electrochimica Acta 188 (2016) 277-285 (Literature 1).

According to the scheme proposed in Literature 1, it has been proposed that, as potential of the working electrode where the electrode reaction is performed increases in the positive direction during the forward scan of cyclic voltammetry, steps I, II, and III take place in sequence. According to the proposal of Patent Literature 1, in step I, glucose is adsorbed onto an electrode surface with simultaneous removal of a hydrogen atom. In step II, the glucose adsorbed on the electrode surface is oxidized to a gluconolactone intermediate adsorbed on the electrode surface. In step III, the gluconolactone intermediate is removed from the electrode surface to become gluconolactone.

With reference to the cyclic voltammogram of Literature 1, the inventors set the potential of the working electrode WE to potential at which steps I and II are performed, apply a voltage to an aqueous solution containing the glucose, and investigated a current value of a current flowing through the working electrode WE.

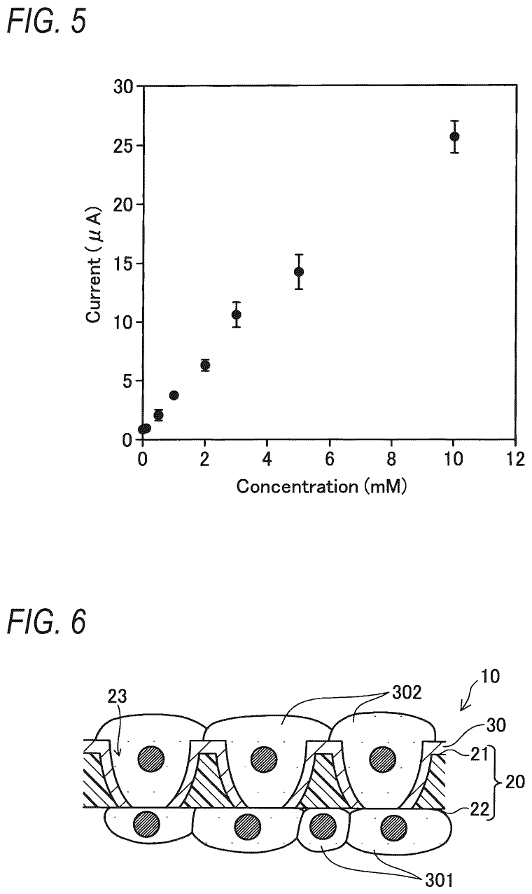

FIG. 5 shows a measurement result obtained by measuring the relationship between the glucose concentration and the current value. Specifically, a plurality of aqueous solutions having various glucose concentrations were prepared, a reference electrode, a counter electrode, and a working electrode were placed in each of the aqueous solutions, and the measurement was performed using a potentiostat. Measurement conditions are as follows.
Reference electrode: Ag/AgCl (sat.KCL)
Counter electrode: Pt
Working electrode: Au (q 3 mm)
Pretreatment: −1.6 V, 1 second
Applied potential: 0.3 V

It should be noted that 0.3 V of the applied potential is the potential of the working electrode at which steps I and II are performed. By measuring a voltage between the reference electrode and the working electrode, a voltage was applied between the working electrode and the reference electrode so that the potential of the working electrode was 0.3 V, and a current value of a current flowing between the working electrode and the counter electrode after 2 seconds from the start of energization was measured. The number of samples is three. The pretreatment is a treatment for making a sueface of the working electrode alkaline. Specifically, hydroxide ions are generated in the vicinity of the working electrode by causing the electrolysis reaction on the working electrode.

As shown in FIG. 5, there is a correlation in which the higher the glucose concentration, the larger the current value. As the glucose concentration in the aqueous solution increases, the number of electrons emitted from the oxidation reaction increases. Therefore, it is expected that the higher the glucose concentration, the larger the current value. This experiment also demonstrated that the higher the glucose concentration, the larger the current value. Thus, by determining the relational expression between the glucose concentration and the current value in advance, the glucose concentration of the aqueous solution can be determined using the measured current value of the aqueous solution.

### B. Another Embodiment of Culture Method

The cell culture using the cell culture membrane 10 in step S20 of FIG. 3 is not limited to the case of cell culture on one face of the cell culture membrane 10, and may be cell culture on both faces of the cell culture membrane 10. In this case, co-culture of culturing two or more different types of cells on both faces of the cell culture membrane 10 may be performed.

FIG. 6 is a schematic diagram when the first cells 301 and second cells 302 of a different type from the first cells 301 are co-cultured on the cell culture membrane 10. The cell culture membrane 10 is attached to, for example, an end portion of an insert accommodated in the well. In a first step of the co-culture using the cell culture membrane 10, the first cells 301 are seeded and cultured on the second face 22 in a state in which the second face 22 of the cell culture membrane 10 is oriented upward in a vertical direction. In the next second step, the second cells 302 are seeded and cultured on the metal film 30 in a state in which the first face 21 of the cell culture membrane 10 is oriented upward in the vertical direction. In the culture, the second cells 302 enter the through holes 23. Thus, the first cell 301 and the second cell 302 can come into contact with each other. For example, by using epithelial cells as the first cells 301 and using vascular endothelial cells as the second cells 302, a three-dimensional model closer to a living body can be produced.

The cell culture using the cell culture membrane 10 is not limited to the above. The same type of cells may be cultured on the first face 21 and the second face 22, or two or more types of cells may be cultured on each of the first face 21 and the second face 22. The cells may be cultured only on the second face 22.

### C. Another Embodiment of Measurement Method

### (C1) Electrochemical Impedance Measurement Using Three Electrodes

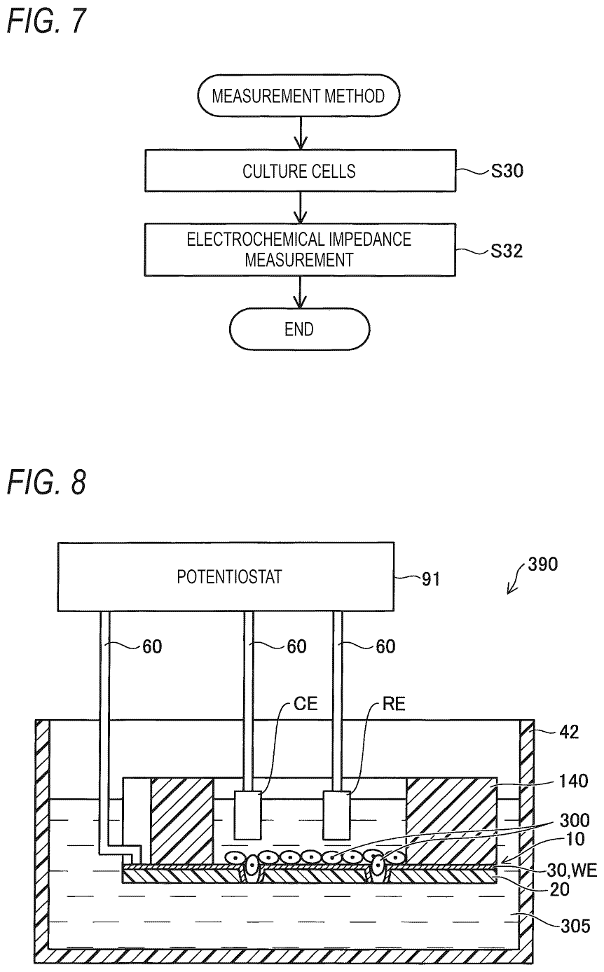

FIG. 7 is a flowchart showing a procedure of a measurement method of measuring impedance. FIG. 8 is a view showing a measurement system 390 for performing electrochemical impedance measurement using three electrodes. The same configurations and processing steps as those of the above embodiment are denoted by the same reference numerals, and detailed description thereof will be appropriately omitted. As shown in FIG. 8, the measurement system 390 includes the potentiostat 91, the counter electrode CE, and the reference electrode RE. The counter electrode CE and the reference electrode RE are electrically connected to the potentiostat 91. The conductive wire 60 electrically connected to the metal film 30 is electrically connected to the potentiostat 91. The counter electrode CE and the reference electrode RE are placed in the culture solution 305 placed inside a cylindrical member 140. The cylindrical member 140 used in the present embodiment is made of a resin and has a cylindrical shape. The cell culture membrane 10 is attached to an end portion of the cylindrical member 140 in the axial direction.

In step S30 as a first step of FIG. 7, the cell culture membrane 10 is immersed in the culture solution 305, and the cells 300 are cultured on the metal film 30 of the cell culture membrane 10. In step S32 as a second step, the electrochemical impedance measurement is performed using the metal film 30 as the working electrode in the culture solution 305. In step S32, the potentiostat 91 is used to control a potential difference between the working electrode WE and the reference electrode RE to be a predetermined potential difference.

Specifically, the impedance of the current path connecting the working electrode WE to the counter electrode CE is obtained. In step S32, an analysis by electrochemical impedance spectroscopy (EIS) may be further performed. Accordingly, information on an interface state of the working electrode WE can be obtained.

### D. Other Embodiments of Cell Culture Membrane

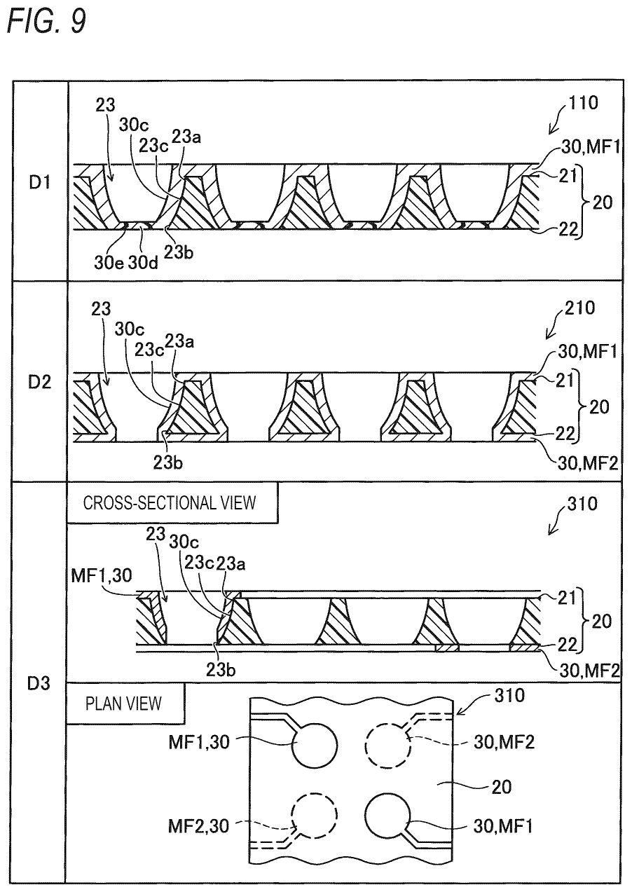

FIG. 9 shows views of embodiments (D1) to (D3), which are other embodiments, of the cell culture membrane 10. The same configurations as those of the above embodiment are denoted by the same reference numerals, and detailed description thereof will be appropriately omitted.

(D1) As shown in “D1” of FIG. 9, a cell culture membrane 110 has the membrane body 20 and the metal film 30. The metal film 30 includes the first metal film MF1. The cell culture membrane 110 is different from the cell culture membrane 10 shown in FIG. 1 in that the first metal film MF1 includes metal closing portions 30d closing the second opening portions 23b of the membrane body 20. The metal closing portion 30d has a crack 30e.

The cell culture membrane 110 is produced similarly to the manufacturing process shown in FIG. 2. However, in step S14, sputtering time is set to be longer than the sputtering time of the cell culture membrane 10. Specifically, sputtering time of the titanium layer 33 is two minutes. Sputtering time of the second metal layer 32 is two minutes. Sputtering time of the first metal layer 31 is nine minutes. Accordingly, a thickness of the metal film 30 of the cell culture membrane 110 is larger than a thickness of the metal film 30 of the cell culture membrane 10. Thus, when the membrane body 20 is removed from the substrate SB in step S16, the metal film formed on the substrate SB is not separated from the membrane body 20 and is maintained in a state of being coupled to the membrane body 20.

The crack 30e is a gap smaller than 3 μm. Since the crack 30e is formed in the cell culture membrane 110, the first cell 301 and the second cell 302 can be brought into contact with each other via the crack 30e when the co-culture is performed. It is expected that the crack 30e can reproduce the role of a fine hole of the base membrane. Therefore, it is expected that a cellular condition closer to that in the living body can be recreated by culture using the cell culture membrane 110.

(D2) As shown in “D2” of FIG. 9, a cell culture membrane 210 has the membrane body 20 and the metal film 30. The metal film 30 includes the first metal film MF1 and a second metal film MF2. The second metal film MF2 is formed to overlap the second face 22 of the membrane body 20. The cell culture membrane 210 is produced similarly to the manufacturing process shown in FIG. 2. However, in step S14, after the metal film 30 is formed by depositing a metal on the first face 21, after the arrangement of the membrane body 20 is changed such that the second face 22 faces upward on the substrate SB, a metal is deposited on the second face 22 to form the second metal film MF2.

(D3) As shown in “D3” of FIG. 9, a cell culture membrane 310 has the membrane body 20 and the metal film 30. The metal film 30 includes the first metal film MF1 and the second metal film MF2. Different from the above (D2), the first metal film MF1 and the second metal film MF2 are formed in one portion of the membrane body 20. In a “plan view” of “D3” in FIG. 9, the first metal film MF1 is indicated by a solid line, and the second metal film MF2 is indicated by a broken line. The first metal film MF1 and the second metal film MF2 are formed at positions away from each other in a membrane face direction. Accordingly, the first metal film MF1 and the second metal film MF2 are not electrically connected. The cell culture membrane 310 is produced similarly to that in the embodiment (D2). However, in step S14, a process of masking is carried out so that the metal film 30 is formed at the desired parts, resulting in the partial formation of the metal film 30. According to this embodiment, when different cells 300 are cultured on the first face 21 and the second face 22 of the cell culture membrane 310, respectively, the impedance can be accurately measured for each cell 300.

The cell culture membrane 10 is not limited to the above. The cell culture membrane 10 may not include the first metal film MF1 and include the second metal film MF2. The first metal film MF1 may not have the metal inner peripheral portion 30c. Since the cell culture membrane 10 has the metal film 30 on a membrane face of the membrane body 20, the electrical measurement can be achieved using the metal film 30 as an electrode.

According to the embodiment described above, the cell culture membrane 10 includes the membrane body 20 and the metal film 30. The metal film 30 includes the first metal film MF1. The membrane body 20 has the plurality of through holes 23. The plurality of through holes 23 have the first average hole diameter at the first face 21 larger than the second average hole diameter at the second face 22. Accordingly, when the cells 300 are cultured on the metal film 30 of the cell culture membrane 10, since the metal film 30 is in contact with or close to the cells 300, a measurement accuracy of a culture state in the vicinity of the cells 300 can be improved. Further, since the cells 300 can enter the through holes 23, when different types of cells 300 are cultured on the first face 21 and the second face 22, the cells cultured on the first face 21 and the cells cultured on the second face 22 can be brought into contact with each other through the respective through holes 23. Thus, the cellular condition closer to that in the living body can be created.

Each through hole 23 has the first opening portion 23a opening at the first face 21, the second opening portion 23b opening at the second face 22, and the inner peripheral portion 23c connecting the first opening portion 23a to the second opening portion 23b. The first metal film MF1 has the metal inner peripheral portion 30c. Accordingly, since it is possible to increase an area of the metal film 30 in contact with or close to the cells 300 that have entered the through holes 23, the measurement accuracy can be further improved.

The membrane body 20 is made of polyurethane. Accordingly, the membrane body 20 having the plurality of through holes 23 whose first average hole diameter is larger than the second average hole diameter can be easily produced. The second average hole diameter is 7 μm or less. Therefore, when the cells 300 having a size of about 10 μm are cultured, it is possible to suppress the wrap-around of the cells 300.

The metal film 30 includes the first metal layer 31 mainly made of gold and the second metal layer 32 mainly made of platinum. The second metal layer 32 is disposed between the first metal layer 31 and the membrane body 20. Accordingly, the measurement accuracy can be improved. The metal film 30 further includes the titanium layer 33. The titanium layer 33 is disposed between the second metal layer 32 and the membrane body 20. Accordingly, the adhesion of the first metal layer 31 and the second metal layer 32 to the membrane body 20 cam be improved.

The measurement method also includes step S20 as the first step of culturing the cells 300 on the metal film 30, and steps S22 and S24 as the second step of measuring the glucose concentration. Accordingly, when the cells 300 are cultured on the metal film 30 of the cell culture membrane 10, the measurement of the glucose concentration including the electrical measurement using the metal film 30 as the electrode can be achieved.

According to the other embodiment (D1), the first metal film MF1 has the metal closing portions 30d closing the second opening portions 23b. The metal closing portion 30d has the crack 30e. Accordingly, when the first cell 301 is cultured on the first face 21 and the second cell 302 is cultured on the second face 22, since the first cell 301 and the second cell 302 can be brought into contact with each other through the crack 30e, the cellular condition closer to that in the living body can be created.

Further, according to the other embodiment (C1), the method includes step S30 as the first step of culturing the cells 300 on the metal film 30, and step S32 as the second step of performing the electrochemical impedance measurement. Accordingly, when the cells 300 are cultured on the metal film 30 of the cell culture membrane 10, the impedance measurement using the metal film 30 as the electrode can be achieved.

### E. EXAMPLES

### E-1. Culture Results

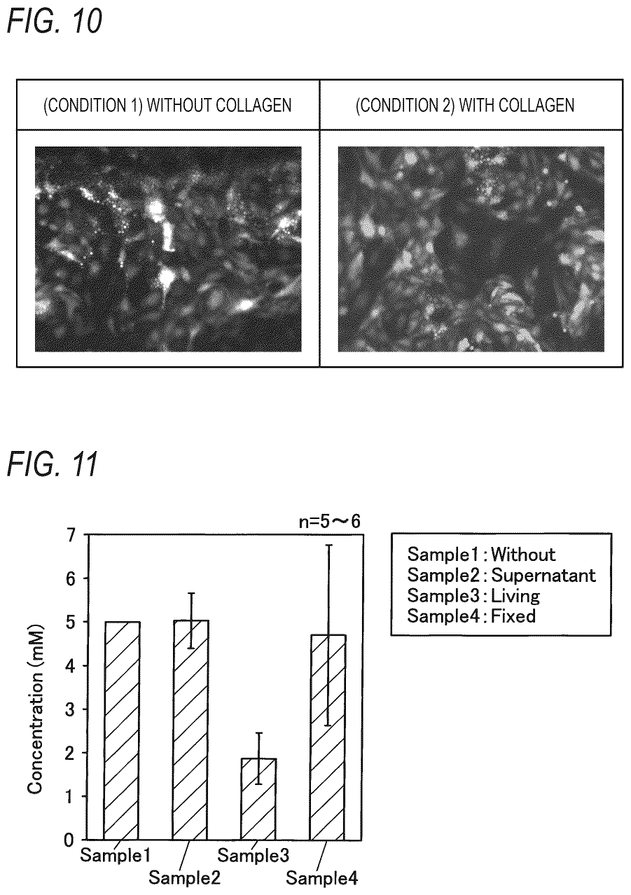

FIG. 10 shows scanning electron microscope (SEM) images obtained by observing the cell culture membrane 10 after the cells were cultured on the metal film 30 of the cell culture membrane 10. The culture was performed under two conditions: “condition 1” in which the cell culture membrane 10 was not collagen-coated and “condition 2” in which the cell culture membrane 10 was collagen-coated. Culture conditions were as follows.
Cell: GFP-HUVEC (Angio-Proteomie)
Medium: endothelial cell growth medium (Promocell, model number: C-22111)
Sowing density: 2.0×10⁴ cells/cm²
(Condition 1) Number of days of culture: 2 days
(Condition 2) Number of days of culture: 4 days

As shown in FIG. 10, cells could be cultured well in either the case where collagen coating was performed or the case where collagen coating was not performed.

### E-2. Measurement of Glucose Concentration in Spheroid

FIG. 11 is a diagram showing measurement results obtained by measuring the glucose concentration for samples 1 to 4. Sample 1 (Without) was PBS with a glucose concentration of 5 mM. Sample 2 (Supernatant) was a medium supernatant obtained after the spheroids were cultured in PBS with a glucose concentration of 5 mM using a culture substrate different from the cell culture membrane 10. Sample 3 (Living) was a medium obtained after the spheroids were cultured in PBS with a glucose concentration of 5 mM using the cell culture membrane 10. Sample 4 (Fixed) was prepared in the following procedure. First, spheroids were cultured using a culture substrate different from the cell culture membrane 10, and the cells were fixed using 4% paraformaldehyde-phosphate buffer solution. Thereafter, immobilized spheroids were transferred to the cell culture membrane 10, and the cell culture membrane 10 with the immobilized spheroids was placed in the PBS having a glucose concentration of 5 mM, and then the collected PBA was used as Sample 4 (Fixed). Cells used in Samples 2 to 4 were MCF-7 (10,000 cells), and the number of days of culturing was 3 days.

The measurement conditions of the glucose concentrations of Samples 1 to 4 were as follows.
Pretreatment: −1.6 V, 1 s
Potential of working electrode: 0.3 V

As shown in FIG. 11, the glucose concentration of Sample 3 was about 2 mM and was decreased from 5 mM. This is because the cells consumed glucose through metabolism. In this regard, in Sample 2, the glucose concentration also decreased because the cells consumed glucose through metabolism, but the amount of decrease was not reflected in the measurement results. This is considered to be because, in the process of placing the medium supernatant into the cylindrical member 40 of the measurement system 90 for measurement, the glucose concentration became almost uniform over the entire aqueous solution, and the decrease amount of the glucose concentration was about the same as the measurement error. As can be seen from comparison between Sample 2 and Sample 3, in Sample 3, minute changes in the glucose concentration in the vicinity of the cells could be measured. According to the cell culture membrane 10, since the metal film 30 was in contact with or close to the cells, it was confirmed that the glucose concentration could be accurately measured in the vicinity of the cells.

The present disclosure is not limited to the above embodiments, and may be implemented by various configurations without departing from the gist of the present disclosure. For example, technical features in the embodiments corresponding to technical features in aspects described in the summary of the invention can be replaced or combined as appropriate to solve a part or all of the above-described problems or to achieve a part or all of the above-described effects. The technical features may be appropriately deleted unless being described as necessary in the present specification.

### REFERENCE SIGNS LIST

10, 110, 210, 310 cell culture membrane
20 membrane body
21 first face
22 second face
23 through hole
23a first opening portion
23b second opening portion
23c inner peripheral portion
30 metal film
30c metal inner peripheral portion
30d metal closing portion
30e crack
31 first metal layer
32 second metal layer
33 titanium layer
40, 140 cylindrical member
42 well
50 resin container
60 conductive wire
80 culture container
90, 390 measurement system
91 potentiostat
300 cell
301 first cell
302 second cell
305 culture solution
CE counter electrode
Da first hole diameter
Db second hole diameter
MF1 first metal film
MF2 second metal film
RE reference electrode
SB substrate
WE working electrode

What is claimed is:

1. A cell culture membrane comprising:
a membrane body having a first face, a second face located on a side opposite to the first face, and a plurality of through holes penetrating from the first face to the second face; and
a metal film including at least one of a first metal film provided to overlap the first face or a second metal film provided to overlap the second face, wherein
the plurality of through holes have a first average hole diameter at the first face larger than a second average hole diameter at the second face.

2. The cell culture membrane according to claim 1, wherein
each of the plurality of through holes has a first opening portion opening at the first face, a second opening portion opening at the second face, and an inner peripheral portion connecting the first opening portion to the second opening portion,
the metal film does not include the second metal film and includes the first metal film, and
the first metal film has a metal inner peripheral portion provided to cover the inner peripheral portion.

3. The cell culture membrane according to claim 2, wherein
the first metal film has a metal closing portion which closes the second opening portion, and
the metal closing portion has a crack.

4. The cell culture membrane according to claim 1, wherein
the membrane body comprises polyurethane.

5. The cell culture membrane according to claim 1, wherein
the second average hole diameter is 7 μm or less.

6. The cell culture membrane according to claim 1, wherein
the metal film includes a first metal layer mainly made of one of gold or platinum.

7. The cell culture membrane according to claim 6, further comprising:
a titanium layer disposed between the first metal layer and the membrane body and mainly made of titanium.

8. The cell culture membrane according to claim 1, wherein
the metal film includes
a first metal layer mainly made of gold, and
a second metal layer mainly made of platinum and disposed between the first metal layer and the membrane body.

9. The cell culture membrane according to claim 8, wherein
the metal film further includes a titanium layer disposed between the second metal layer and the membrane body and mainly made of titanium.

10. A measurement method comprising:
immersing the cell culture membrane according to claim 1 in a culture solution to culture a cell on the metal film of the cell culture membrane; and
electrochemically measuring a glucose concentration using the metal film as a working electrode in the culture solution.

11. A measurement method comprising:
immersing the cell culture membrane according to claim 1 in a culture solution to culture a cell on the metal film of the cell culture membrane; and
performing electrochemical impedance measurement using the metal film as a working electrode in the culture solution.
